(19) United States
(12) Patent Application Publication
(10) Pub. No.: US 2025/0298112 A1
(43) Pub. Date: Sep. 25, 2025

(54) Characterization of Concomitant Field Effects on MR Imaging

(71) Applicants: Siemens Healthineers AG, Forchheim (DE); Centre National de la Recherche Scientifique (CNRS), Paris (FR); Institut National de La Sante et de la Recherche Medicale (INSERM), Paris (FR); King's College London, London (GB); Universite Paris XIII Paris-Nord, Villetaneuse (FR); Université Paris Cité, Paris (FR)

(72) Inventors: Omar Darwish, London (GB); Radhouene Neji, London (GB); Ralph Sinkus, Parmain (FR)

(73) Assignees: Siemens Healthineers AG, Forchheim (DE); Centre National de la Recherche Scientifique (CNRS), Paris (FR); Institut National de La Sante et de la Recherche Medicale (INSERM), Paris (FR); King's College London, London (GB); Universite Paris XIII Paris-Nord, Villetaneuse (FR); Université Paris Cité, Paris (FR)

(21) Appl. No.: 19/081,196

(22) Filed: Mar. 17, 2025

(30) Foreign Application Priority Data
Mar. 19, 2024 (EP) 24164455.8

(51) Int. Cl. G01R 33/565; A61B 5/055; G01R 33/385

(52) U.S. Cl. CPC G01R 33/56581; A61B 5/055; G01R 33/385

(57) ABSTRACT

The present disclosure relates to a method of performing 3D Magnetic Resonance Imaging including applying a magnetic gradient field that causes a concomitant field B_{c}. A further step of the method includes determining phase accruals due to the self-squared terms of the concomitant field B_{c} and phase accruals φ_{xz}, φ_{yz} due to the cross terms of the concomitant field B_{c} based on an encoding matrix that accounts for the different possible sign combinations of the applied magnetic gradients.

### CROSS-REFERENCE TO RELATED APPLICATIONS

The present application claims priority to and the benefit of European patent application no. EP 24164455.8, filed on Mar. 19, 2024, the contents of which are incorporated herein by reference in their entirety.

### TECHNICAL FIELD

The present disclosure relates to a method of performing 3D Magnetic Resonance Imaging (3D MRI, such as a 3D Magnetic Resonance Elastography (3D MRE) including applying a magnetic gradient field and determining phase accruals. Furthermore, the present disclosure relates to a respective 3D magnetic resonance imaging system and a computer program, as well as a computer-readable medium.

### BACKGROUND

Hepatic three-dimensional MR Elastography (3D MRE) has shown promises for measuring liver fibrosis and grading liver inflammation using viscoelastic parameters derived from the 3D displacement field as discussed in Shi Y, Qi Y F, Lan G Y, et al. Three-dimensional MR Elastography Depicts Liver Inflammation, Fibrosis, and Portal Hypertension in Chronic Hepatitis B or C. Radiology. October 2021; 301(1):154-162, doi:10.1148/radiol.2021202804 and in Darwish O I, Gharib A M, Jeljeli S, et al. Single Breath-Hold 3-Dimensional Magnetic Resonance Elastography Depicts Liver Fibrosis and Inflammation in Obese Patients. Invest Radiol, Jun. 1, 2023; 58(6):413-419, doi:10.1097/rli.0000000000000952.

Fibrosis is measured using the shear wave speed (Cs [m/s]) and inflammation is graded with the loss modulus (G″ [kPa]) (compare Sinkus R, Lambert S, Abd-Elmoniem K Z, et al. Rheological determinants for simultaneous staging of hepatic fibrosis and inflammation in patients with chronic liver disease. NMR Biomed. October 2018; 31(10):e3956, doi:10.1002/nbm.3956).

Hepatic 3D MRE has been limited to high field MR systems (B0≥1.5 T). Nonetheless, expanding hepatic 3D MRE to wide-bore low field MR systems (B0≤1.0 T) can serve as a means of accommodating liver patients with high BMI and/or claustrophobia such as non-alcoholic fatty liver disease (NAFLD) patients. NAFLD is a growing global healthy crisis and enabling access to 3D MRE for NAFLD patients is becoming of importance in the clinic. Furthermore, the longer T2* relaxation times at low field may be beneficial to mitigate iron overload in NAFLD patients. Another aspect to consider is that low field MR system bring down the financial entry point of MR which might allow a wider spread of hepatic 3D MRE in middle-income countries.

However, low field MR systems come with a penalty in signal-to-noise ratio (SNR), which is directly proportional to a penalty in phase-to-noise ratio (PNR) in 3D MRE. A Hadamard motion encoding scheme may be used to increase sensitivity to motion, thereby mitigating the decrease in PNR (compare Guenthner C, Runge J H, Sinkus R, Kozerke S. Analysis and improvement of motion encoding in magnetic resonance elastography; NMR Biomed, May 2018; 31(5):e3908. doi:10.1002/nbm.3908).

Hadamard motion encoding applies unique combinations of motion encoding gradients on all the gradient axes simultaneously; this however leads to concomitant fields, which are higher with a decreasing static magnetic field B0 (Bernstein M A, Zhou X J, Polzin J A, et al. Concomitant gradient terms in phase contrast MR: Analysis and correction. Magnetic Resonance in Medicine, 1998; 39(2):300-308, doi: https://doi.org/10.1002/mrm.1910390218).

### SUMMARY

The object of the present disclosure is to provide a 3D MRI method and device with improved phase-to-noise ratio.

According to the present disclosure, this object is solved by a method, system, computer program, and computer-readable medium as described herein in accordance with the various embodiments, including the claims.

Accordingly, there is provided a method of performing 3D Magnetic Resonance Imaging including the steps of:
applying a magnetic gradient field that causes a concomitant field B_{c} leading to a phase accrual as represented in Equation 1 below:

$${φ}_{c} = \frac{γ T}{2 {B}_{0}} ( {\overbrace{{G}_{x}^{2} {z}^{2} + {G}_{y}^{2} {z}^{2} + {G}_{z}^{2} \frac{{x}^{2} + {y}^{2}}{4}}}^{“ self - squared ” terms} {\underbrace{- {G}_{x} {G}_{z} x z - {G}_{y} {G}_{z} y z}}_{“ cross ” terms} ) \qquad Eqn . 1$$

the terms including Gₓ², G_{y}² and G_{z}² are called self-squared terms and the terms including GₓG_{z} and G_{y}G_{z} are called cross terms, wherein x, y and z are coordinates of a 3D space, B₀ is a static magnetic field, Gₓ, G_{y} and G_{z} are (interleaved) applied magnetic gradients, γ is the gyromagnetic ratio characteristic of the nuclei and T is the total time duration for applying the magnetic gradients, and
determining phase accruals due to the self-squared terms of the concomitant field B_{c} and phase accruals φ_{xz}, φ_{yz} due to the cross terms of the concomitant field B_{c} based on an encoding matrix that accounts for the different possible sign combinations of the applied magnetic gradients.

Whenever a desired linear magnetic field gradient is activated, additional magnetic fields with nonlinear spatial dependence result (compare Bernstein et al.). This is a consequence of Maxwell's Equations for the divergence and curl of the magnetic field. The above-formulated concomitant field B_{c} is the consequence. The concomitant field affects the phase contrast. Specifically, the phase accumulated by transverse magnetization subjected to the concomitant field is represented in accordance with Eqn. 2 below as follows:

$${φ}_{c} ( x , y , z ) = γ \int {B}_{c} ( x , y , z , t ) dt \qquad Eqn . 2$$

wherein γ represents the gyromagnetic ratio characteristic of the nuclei.

Advantageously, such a 3D MRI/MRE method may be implemented. Furthermore, MRI/MRE measurements are possible on all fields strengths. There is no limitation to 0.55 T or Hadamard motion encoding. Specifically, there can be provided a rapid 3D slap-selective MR elastography using interleaved motion encoding.

In an embodiment, MRE phase measurements for determining the phase accruals are performed according to an encoding scheme represented by a predetermined invertible encoding matrix, which may be invertible. This means that the equation system may be solved unambiguously.

According to a further embodiment, at least or exactly five (MRI or MRE) phase measurements are performed for unambiguously determining the phase accruals due to the self-squared terms of the concomitant field B_{c} and due to the crossterms of the concomitant field B_{c}, and wherein the encoding matrix is a 5×5 matrix. Thus, there is an equation system including five unknowns. Each equation includes a phase measurement result and one phase accrual out of the self-squared terms and the crossterms. If an adequate 5×5 encoding matrix is used, the phase accruals can be determined unambiguously.

In an alternative embodiment at least or exactly six (e.g. MRE) phase measurements m₁ to m₆ are performed for clearly determining the phase accruals φ_{Uz}, φ_{Ux}, φ_{Uy} due to a 3D displacement field and the phase accruals φ_{xz}, φ_{yz} due to the cross terms of the concomitant field B_{c} as well as a phase accrual (er due to a constant phase error and due to the self-squared terms of the concomitant field Bc, and wherein the encoding matrix M is a 6×6 matrix represented in accordance with Equation 3 below as follows:

$$[ \begin{matrix}{m}_{1} \\ {m}_{2} \\ {m}_{3} \\ {m}_{4} \\ {m}_{5} \\ {m}_{6}\end{matrix} ] = M [ \begin{matrix}{φ}_{{U}_{z}} \\ {φ}_{{U}_{x}} \\ {φ}_{{U}_{y}} \\ {φ}_{err} \\ {φ}_{xz} \\ {φ}_{yz}\end{matrix} ] \qquad Eqn . 3$$

Beside the phase accruals due to the crossterms, typically a phase accrual due to a constant phase error and due to the self-squared terms occurs. If this phase error is unknown, it can be integrated into the equation system including six equations and six unknowns. Thus, no more than six phase measurements are necessary to determine the six phase accruals. If a further component of the phase accrual has to be determined, beside the six unknowns, even a 7×7 encoding matrix (or larger) may be used. In this case, at least seven phase measurements are necessary to unambiguously determine the seven unknowns. In other words, at least five phase measurements are performed to determine the phase accruals due to the self-squared terms and the crossterms of the concomitant field B_{c}. If more parameters are to be determined, more than five phase measurements may be performed and higher squared matrixes than 5×5 may be used.

According to a further embodiment, the invertible encoding matrix only includes elements like −1 and +1. This means that motion encoding gradients applied on all gradient axes are bipolar. For instance, the bipolar motion encoding gradient G may include two lopes G₁ and G₂, wherein G₂=−G₁.

In another embodiment, the invertible encoding matrix includes elements that reflect the amplitudes of the applied magnetic gradients. This means that the amplitudes do not always have to be the same among one other.

In a specific embodiment the 6×6 matrix is represented in Equation 4, and is equal to:

$$M = [ \begin{matrix}- 1 & + 1 & - 1 & + 1 & + 1 & - 1 \\ + 1 & - 1 & - 1 & + 1 & + 1 & + 1 \\ - 1 & - 1 & + 1 & + 1 & - 1 & + 1 \\ + 1 & + 1 & + 1 & + 1 & - 1 & - 1 \\ + 1 & - 1 & + 1 & + 1 & + 1 & - 1 \\ - 1 & + 1 & + 1 & + 1 & + 1 & + 1\end{matrix} ] \qquad Eqn . 4$$

or any other matrix having the same rows like M.

This 6×6 encoding matrix is a further development of the 4×4 hadamard matrix. The 6×6 encoding matrix allows for unambiguously solving the self-squared terms, the crossterms, and the phase error. Thus, the respective encoded MRI or MRE measurement includes unique combinations of motion encoding gradients applied on all gradient axes simultaneously with equal amplitudes. Specifically, the 6×6 encoding matrix accounts for the different possible sign combinations of the crossterms. The same results can be obtained by using matrices having the same rows like M, but in a different order.

According to an embodiment, the magnetic gradients Gₓ, G_{y} and G_{z} have equal amplitudes. For instance, the gradients of the above 6×6 matrix are equal in amplitude and have different signs. However, similar results may be obtained by using gradient amplitudes different from each other. Furthermore, two of the gradients may have the same amplitude, whereas the third gradient may have a different amplitude. Such an encoding scheme may be used to emphasize one spacial direction.

According to a further embodiment, the motion encoding gradients are applied without any overlap with the imaging gradients. Thus, the motion encoding gradients are temporarily separated from the imaging gradients, thereby obtaining unambiguous stimulations.

In another embodiment, a 2D or 3D image is generated and optionally displayed based on one of the phase accruals φ_{Uz}, φ_{Ux}, φ_{Uy}, φ_{xz}, φ_{yz}. Such images provide information about the elasticity and viscosity of an object under investigation.

According to a further embodiment, a 2D or 3D image is generated and optionally displayed based on an amplitude of a temporal Fourier transform of the phase accruals φ_{Uz}, φ_{Ux}, φ_{Uy}, φ_{xz}, φ_{yz}. The Fourier transforms show the spectral distribution of the phase accruals at a specific mechanical vibration frequency (e.g. 60 Hz).

In an embodiment, any of the above methods is performed as part of a 3D Magnetic Resonance Elastography (MRE). Thus, concomitant field effects may be quantified and assessed in MR Elastography. However, the above methods may be used for other MR techniques as well, where additional gradients are applied besides the imaging gradients.

In an embodiment, the mechanical excitation may performed at any suitable frequency, such as one within the range of 20 to 100 Hz, 20 to 60 Hz, etc., and which may include a 60 Hz or other suitable frequency. Such frequencies enable high quality in MRE measurements of human tissue.

The above object is also solved by a 3D Magnetic Resonance Imaging (MRI) system including a magnet device for applying magnet gradient field and a computing unit for determining phase accruals, wherein the system is configured to perform a method as described above. Specifically, there may be provided a 3D MRE system including the above magnet device and computing unit as well as an actuator for generating shear waves.

Moreover, there is provided a computer program comprising instructions which, when the program is executed by a 3D magnetic resonance imaging system, cause the magnetic resonance imaging system to carry out the method described above. Furthermore, there may be provided a computer-readable medium comprising instructions, which, when executed by a 3D magnetic resonance imaging system, cause the magnetic resonance imaging system to carry out the method described above.

### BRIEF DESCRIPTION OF THE DRAWINGS

The present disclosure will now be described in more detail in connection with the attached drawings showing in:

FIG. 1 illustrates a schematic diagram of an example MRE system, in accordance with the disclosure;

FIG. 2 illustrates example unwrapped phases and Fourier transforms of phantom measurements without mechanical excitation, in accordance with the disclosure; and

FIG. 3 illustrates example unwrapped phases and Fourier transforms of the phantom measurement with mechanical excitation, in accordance with the disclosure.

### DETAILED DESCRIPTION OF THE DISCLOSURE

The following embodiments represent preferable examples of the present disclosure.

The following embodiments relate to MRE systems and methods. However, they can be used for other MR techniques too where additional gradients are applied besides the imaging gradients.

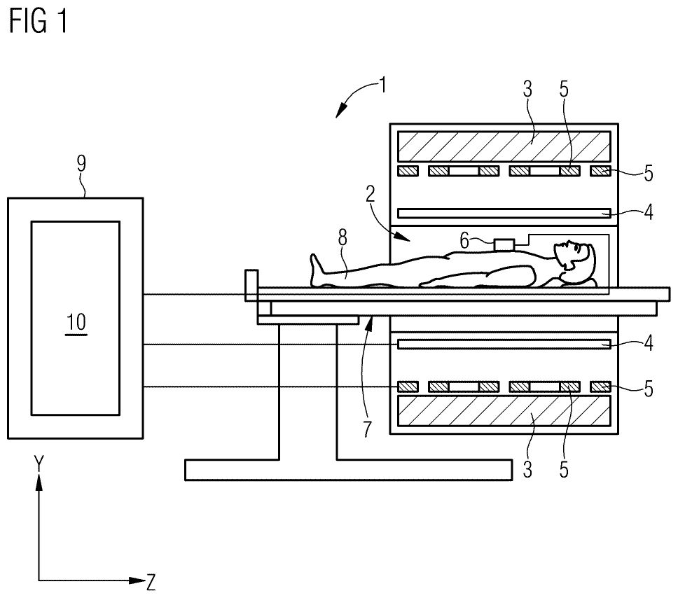

FIG. 1 illustrates a schematic diagram of an example MRE system, in accordance with the disclosure.

The MRE-system comprises a magnet unit with an imaging region 2, for example within a patient tunnel for placing an object 8, e.g. a patient, to be imaged. The magnet unit comprises a field magnet 3 (also referred to herein as a main magnet) that generates a main magnet field for aligning nuclear spins of the object, e.g. within the imaging region 2. The imaging region 2 is characterized by a very homogeneous static main magnet field, the homogeneity relating, e.g. to the magnetic field strength. The field magnet 3 may, for example, be a superconducting magnet capable of providing magnetic fields with a magnetic flux density in the order of several Tesla, e.g. in the order of 7 T or more. A patient table 7 may be movable within the patient tunnel.

Furthermore, the magnet unit comprises a gradient coil arrangement 5 with several gradient coils that are designed to superimpose location-dependent magnetic fields in the three spatial directions on the static main magnet field for spatial differentiation and e.g. slice selection. The gradient coils of the gradient coil arrangement 5 may, for example, be designed as coils of normal conducting wires, which may, for example, generate mutually orthogonal fields or field gradients in the recording region.

The MRE-system 1, e.g. the magnet unit, comprises a transmission coil arrangement, which contains one or more RF-coils 4. It is noted that the one or more RF-coils 4 of the transmission coil arrangement may, depending on the specific implementation or application, also be used as receiving coils. Optionally, the MRE-system 1 may also comprise one or more local coils (not shown in FIG. 1), which may be arranged in the immediate vicinity of the object 8, for example on the object 8 or in the patient table 7. The local coils may serve as receiving coils and/or transmission coils.

Furthermore, the MRE-system comprises a (pulse) wave generator 6 for exciting shear waves within the object/patient to be examined.

The MRE-system 1 also comprises a data processing apparatus 9 including at least one computing unit 10 (also referred to herein as a controller, processing circuitry, or an evaluation device). The at least one computing unit 10 is configured to carry out a computer-implemented method for controlling the MRE-system according to a specific timing scheme. For instance, the time control of the wave generator 6 may be synchronized with the MR imaging. As a result of the computer-implemented method, the at least one computing unit 10 controls the of the MRE-system so that the acquisition is performed in a very efficient way.

In response to the excitation RF-pulses, the at least one computing unit 10 receives corresponding NMR-signals from the receiving coils and may generate respective MR-images of the object 8 depending on those signals.

For instance, the at least one computing unit may comprise a readout control unit, which is connected to the at least one RF-coil 4 and/or the local coil. Depending on the detected MR-signals, the readout control unit, which may comprise an analog-to-digital converter (ADC), may generate corresponding MR-data, e.g. in k-space.

The at least one computing unit 10 may evaluate the MR-data and, for example, carry out a two-dimensional or three-dimensional image reconstruction based on the MR-data. The at least one computing unit 10 also comprises a sending control unit, which is connected to and controls the RF-coil(s) 4 and/or the local coil to emit the excitation RF-pulses and, for example refocusing RF-pulses and other RF-pulses. The at least one computing unit 10 comprises gradient control unit, which is connected to and controls the gradient coil arrangement 5 to apply, for example, slice selecting gradients, gradients for frequency and/or phase encoding, defocusing gradients, and/or readout gradients, and so forth. Additionally, the at least one computing unit 10 comprises a wave generation control unit adapted to control the wave generator.

It is noted that the described structure of the MRE-system 1 is a non-limiting example only. The different required tasks and functions may also be distributed differently and/or to different units in other applications.

Concomitant field effects in MRE might become of importance when translating MRE to low field MR systems. To design an experiment that measures the effect of concomitant fields B_{c} on Hadamard-encoded 3D MRE, we first look at the phase accrual φ_{c} of B_{c} following the work of Bernstein et al.:

$${φ}_{c} = \frac{γ T}{2 {B}_{0}} ( {\overbrace{{G}_{x}^{2} {z}^{2} + {G}_{y}^{2} {z}^{2} + {G}_{z}^{2} \frac{{x}^{2} + {y}^{2}}{4}}}^{“ self - squared ” terms} {\underbrace{- {G}_{x} {G}_{z} x z - {G}_{y} {G}_{z} y z}}_{“ cross ” terms} ) \qquad Eqn . 1$$

A Hadamard-encoded MRE measurement consists of unique combinations of motion encoding gradients applied on all gradient axes simultaneously with equal amplitudes. The following 4×4 Hadamard matrix (H) is typically used in MRE measurement in accordance with Equation 5:

$$H = [ \begin{matrix}- 1 & + 1 & - 1 & + 1 \\ + 1 & - 1 & - 1 & + 1 \\ - 1 & - 1 & + 1 & + 1 \\ + 1 & + 1 & + 1 & + 1\end{matrix} ] \qquad Eqn . 5$$

The first three terms in Eqn. 1, the “self-squared” terms, are constant throughout the different measurements of Hadamard encoding and are therefore encoded in the same term that encodes constant phase errors such as magnetic field inhomogeneity. However, the last two terms, the “cross” terms, change signs between the different measurements of Hadamard encoding depending on the polarity of the applied motion encoding gradients. As a result, the “cross” terms cannot be resolved using a conventional 4×4 Hadamard encoding matrix.

A larger squared encoding matrix (e.g. 5×5, 6×6, 7×7 matrix) can resolve the “cross” terms.

For instance, the Hadamard encoding can be extended to a 6×6 encoding matrix M that accounts for the different possible sign combinations of the “cross” terms, as shown in Equations 3 and 4:

$$M = [ \begin{matrix}- 1 & + 1 & - 1 & + 1 & + 1 & - 1 \\ + 1 & - 1 & - 1 & + 1 & + 1 & + 1 \\ - 1 & - 1 & + 1 & + 1 & - 1 & + 1 \\ + 1 & + 1 & + 1 & + 1 & - 1 & - 1 \\ + 1 & - 1 & + 1 & + 1 & + 1 & - 1 \\ - 1 & + 1 & + 1 & + 1 & + 1 & + 1\end{matrix} ] \qquad Eqn . 4$$

$$[ \begin{matrix}{m}_{1} \\ {m}_{2} \\ {m}_{3} \\ {m}_{4} \\ {m}_{5} \\ {m}_{6}\end{matrix} ] = M [ \begin{matrix}{φ}_{{U}_{z}} \\ {φ}_{{U}_{x}} \\ {φ}_{{U}_{y}} \\ {φ}_{err} \\ {φ}_{xz} \\ {φ}_{yz}\end{matrix} ] \qquad Eqn . 3$$

Where mₖ (k=1, 2, . . . , 6) represents the k-th MRE phase measurement, φ_{U}_{ₓ}, φ_{U}_{_{y}}, and φ_{U}_{_{z}} represent the phase accruals due to the 3D displacement field, φₑᵣᵣ represents the phase accrual due to constant phase errors and the phase accrual of the “self-squared” terms, and φ_{xz} and φ_{yz} represent the phase accrual due to the first and second “cross” terms, respectively.

The proposed encoding matrix M is incorporated into a previously published 3D MRE sequence (Guenthner et al.) extending the sequence from 4 to 6 measurements. The motion encoding gradients are applied without any overlap with the imaging gradients.

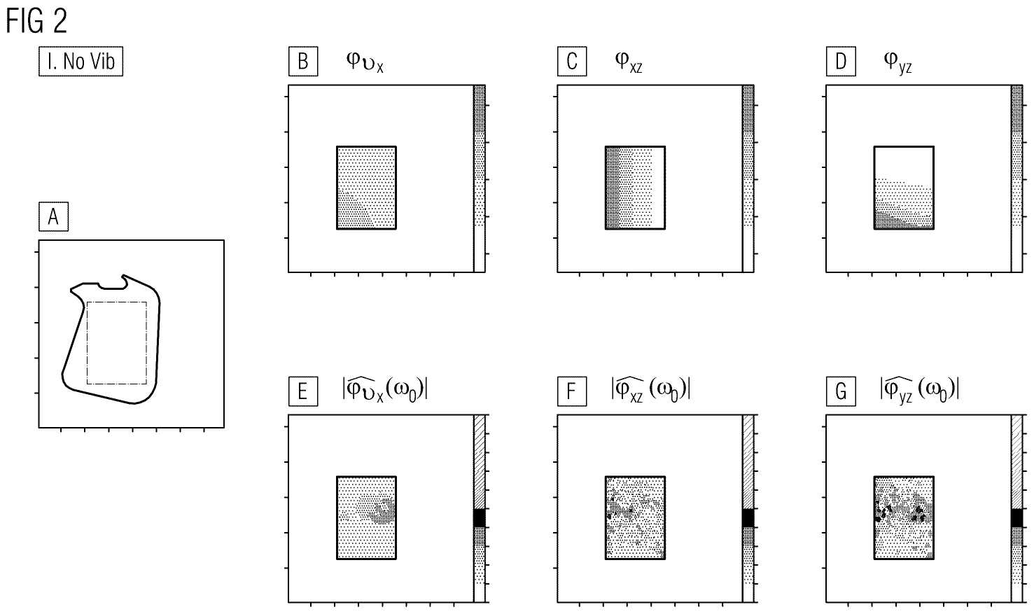

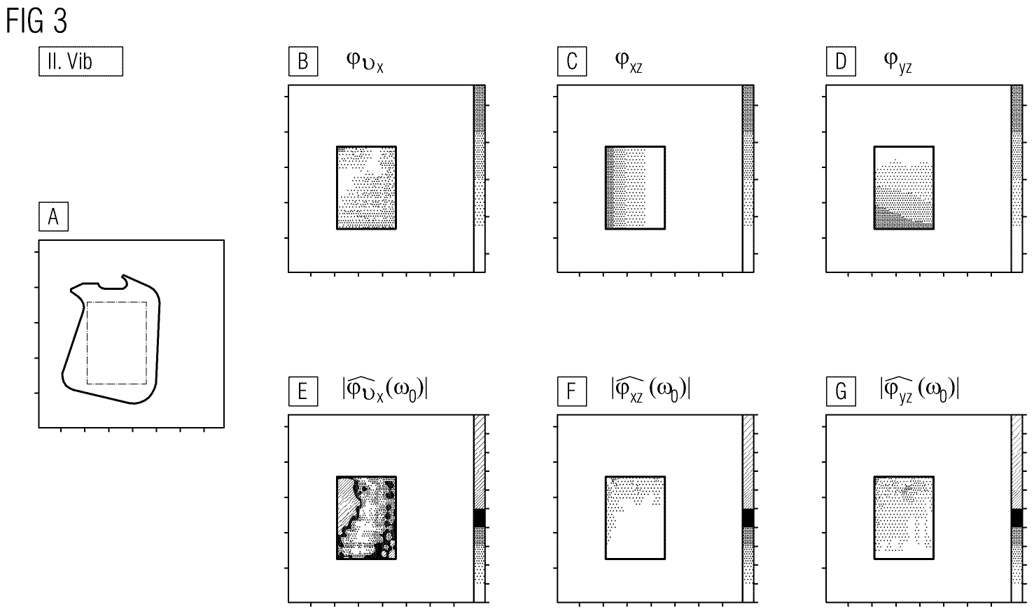

The extended 3D MRE sequence can be implemented on a 0.55 T system. Two phantom experiments are conducted: (I) without vibration to verify that M is solving for the “cross” terms (see FIG. 2). Note that the first “cross” term (GₓG_{z}xz) varies with x and z, and the second “cross” term (G_{y}G_{z}yz) varies with y and z, both independent of the vibration. (II) an experiment with 60 Hz mechanical excitation (see FIG. 3) to examine the phase accrual due to B_{c} with respect to the acquired mechanical wave phase offsets. The phantom used is an ultrasound gel phantom shifted to right of the iso-centre of the bore to increase the apparent effect of the concomitant fields.

The results of the phantom experiment without mechanical excitation, i.e. without vibrations, are shown in FIG. 2. There is no spatial variation in φ_{U}_{ₓ} (FIG. 1.B). However, we can observe a spatial variation in x (left-right direction) in φ_{xz} (FIG. 2.C) and a spatial variation in y (posterior-anterior direction) in φ_{yz} (FIG. 2.D). The spatial variation observed in FIG. 2.C-D suggests that the proposed encoding matrix M is solving for the “cross” terms. The amplitude of the displacement field in the three terms φ_{U}_{ₓ}, φ_{xz}, φ_{yz}) is very low (0.9±0.2 um, 1.4±0.2 um, and 1.6±0.2 um respectively) (FIG. 2.E-G).

Specifically, FIG. 2.A shows a magnitude image calculated by averaging the central four slices of the acquisition volume. FIG. 2.B-D show the unwrapped phases φ_{U}_{ₓ}, φ_{xz}, and φ_{yz}) after decoding of a single slice, and FIG. 2.E-G show the amplitude of the temporal Fourier transform ( [symbol] , [symbol] , [symbol] ) at the mechanical vibration frequency (φ₀=60 Hz) of φ_{U}_{ₓ}, φ_{xz}, and φ_{yz}, respectively averaged over the central four slices.

The results of the phantom experiments with mechanical excitation are presented in FIG. 3. φ_{U}_{ₓ} (FIG. 3.B) varies with the mechanical excitation, however, φ_{xz} (FIG. 3.C) and φ_{yz} (FIG. 3.D) only vary spatially in x (left-right direction) and in y (posterior-anterior direction) respectively. In addition, the amplitude of the displacement field in O_{U}_{ₓ} (81±11 um) is approximately an order of magnitude higher than the amplitudes in φ_{xz} (5±2 um) and φ_{yz} (10±2 um) (FIG. 3.E-G).

Specifically, FIG. 3.A shows a magnitude image calculated by averaging the central four slices of the acquisition volume. FIG. 3.B-D show the unwarped phases (φ_{U}_{ₓ}, φ_{xz}, and φ_{yz}) after decoding of a single slice, and FIG. 3.E-G show the amplitude of the temporal fourier transform ( [symbol] , [symbol] , [symbol] ) at the mechanical vibration frequency (ω₀=60 Hz) of φ_{U}_{ₓ}, φ_{xz}, and φ_{yz} respectively averaged over the central four slices.

The embodiments described herein method may be extended to other MRE measurements on all field strengths, and is not limited only to 0.55 T systems or to Hadamard motion encoding.

The various components described herein may be referred to as “devices” or “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such units and/or devices, as applicable and relevant, may alternatively be referred to herein as “circuitry,” “controllers,” “processors,” or “processing circuitry,” or alternatively as noted herein.

What is claimed is:

1. A method for performing 3D magnetic resonance imaging, comprising:
applying a magnetic gradient field that causes a concomitant field B_{c} leading to a phase accrual φ_{c} represented as:
$${φ}_{c} = \frac{γ T}{2 {B}_{0}} ( \overbrace{{G}_{x}^{2} {z}^{2} + {G}_{y}^{2} {z}^{2} + {G}_{z}^{2} \frac{{x}^{2} + {y}^{2}}{4}} \underbrace{- {G}_{x} {G}_{z} x z - {G}_{y} {G}_{z} y z} ) ,$$
the terms Gₓ², G_{y}² and G_{z}² comprise self-squared terms,
the terms GₓG_{z} and G_{y}G_{z} comprise cross terms,
x, y and z represent coordinates in a 3D space,
B₀ represents a static magnetic field,
Gₓ, G_{y} and G_{z} represent applied magnetic gradients,
γ represents a gyromagnetic ratio characteristic of nuclei,
T represents a total time duration for applying the magnetic gradients;
determining phase accruals φ_{xz}, φ_{yz} due to the self-squared terms of the concomitant field B_{c} and due to the cross terms of the concomitant field B_{c}, based on an encoding matrix that accounts for different possible sign combinations of the applied magnetic gradients Gₓ, G_{y} and G_{z}; and
generating a 2D or 3D image based upon one or more of the phase accruals φ_{xz}, φ_{yz}.

2. The method according to claim 1, further comprising:
performing phase measurements for determining the phase accruals according to an encoding scheme represented by a predetermined invertible encoding matrix.

3. The method according to claim 2, wherein the performing the phase measurements comprises performing five phase measurements for determining the phase accruals based upon the self-squared terms of the concomitant field B_{c} and the cross terms of the concomitant field B_{c}, and
wherein the predetermined invertible encoding matrix is a 5×5 matrix.

4. The method according to claim 2, further comprising:
performing the phase measurements as six phase measurements represented as m₁ to m₆ for determining:
phase accruals φ_{Uz}, φ_{Ux}, φ_{Uy} based upon to a 3D displacement field,
phase accruals φ_{xz}, φ_{yz} due to the cross terms of the concomitant field B_{c}, and
a phase accrual φₑᵣᵣ based upon to a constant phase error and the self-squared terms of the concomitant field B_{c},
wherein the predetermined invertible encoding matrix M is a 6×6 matrix represented as:
$$[ \begin{matrix}{m}_{1} \\ {m}_{2} \\ {m}_{3} \\ {m}_{4} \\ {m}_{5} \\ {m}_{6}\end{matrix} ] = M [ \begin{matrix}{φ}_{{U}_{z}} \\ {φ}_{{U}_{x}} \\ {φ}_{{U}_{y}} \\ {φ}_{err} \\ {φ}_{xz} \\ {φ}_{yz}\end{matrix} ] .$$

5. The method according to claim 2, wherein the predetermined invertible encoding matrix only includes elements comprising −1 and/or +1.

6. The method according to claim 2, wherein the predetermined invertible encoding matrix includes elements that reflect amplitudes of the applied magnetic gradients Gₓ, G_{y} and G_{z}.

7. The method according to claim 4, wherein the 6×6 predetermined invertible encoding matrix M is represented as:
$$M = [ \begin{matrix}- 1 & + 1 & - 1 & + 1 & + 1 & - 1 \\ + 1 & - 1 & - 1 & + 1 & + 1 & + 1 \\ - 1 & - 1 & + 1 & + 1 & - 1 & + 1 \\ + 1 & + 1 & + 1 & + 1 & - 1 & - 1 \\ + 1 & - 1 & + 1 & + 1 & + 1 & - 1 \\ - 1 & + 1 & + 1 & + 1 & + 1 & + 1\end{matrix} ] .$$

8. The method according to claim 1, wherein the applied magnetic gradients Gₓ, G_{y}, and G_{z} have equal amplitudes.

9. The method according to claim 1, wherein the applied magnetic gradients Gx, Gy, and Gz have unequal amplitudes.

10. The method according to claim 1, further comprising:
applying motion encoding gradients without any overlap with imaging gradients.

11. The method according to claim 4, further comprising:
displaying a 2D or 3D image based on one of the phase accruals φ_{Uz}, φ_{Ux}, φ_{Uy}, φ_{xz}, φ_{yz}.

12. The method according to claim 4, further comprising:
displaying a 2D or 3D image based on an amplitude of a temporal Fourier transform of one of the phase accruals φ_{Uz}, φ_{Ux}, φ_{Uy}, φ_{xz}, φ_{yz}.

13. The method according to claim 1, wherein the 3D magnetic resonance imaging comprises at least part of a 3D magnetic resonance elastography examination.

14. The method according to claim 1, further comprising:
performing mechanical excitation at a frequency in a range of 20 to 100 Hz.

15. The method according to claim 1, further comprising:
performing mechanical excitation at a frequency in a range of 20 to 60 Hz.

16. A 3D magnetic resonance imaging system, comprising:
a magnet configured to apply a magnetic gradient field that causes a concomitant field B_{c} leading to a phase accrual φ_{c} represented as:
$${φ}_{c} = \frac{γ T}{2 {B}_{0}} ( \overbrace{{G}_{x}^{2} {z}^{2} + {G}_{y}^{2} {z}^{2} + {G}_{z}^{2} \frac{{x}^{2} + {y}^{2}}{4}} \underbrace{- {G}_{x} {G}_{z} x z - {G}_{y} {G}_{z} y z} ) ,$$
wherein:
the terms Gₓ², G_{y}² and G_{z}² comprise self-squared terms,
the terms GₓG_{z} and G_{y}G_{z} comprise cross terms,
x, y and z represent coordinates in a 3D space,
B₀ represents a static magnetic field,
Gₓ, G_{y} and G_{z} represent applied magnetic gradients,
γ represents a gyromagnetic ratio characteristic of nuclei,
T represents a total time duration for applying the magnetic gradients; and
processing circuitry configured to:
determine phase accruals φ_{xz}, φ_{yz} due to the self-squared terms of the concomitant field B_{c} and due to the cross terms of the concomitant field B_{c}, based on an encoding matrix that accounts for different possible sign combinations of the applied magnetic gradients Gₓ, G_{y} and G_{z}; and
generate a 2D or 3D image based upon one or more of the phase accruals φ_{xz}, φ_{yz}.

17. A computer-readable medium having instructions stored thereon that, when executed by a 3D magnetic resonance imaging system, cause the magnetic resonance imaging system to:
apply a magnetic gradient field that causes a concomitant field B_{c} leading to a phase accrual φ_{c} represented as:
$${φ}_{c} = \frac{γ T}{2 {B}_{0}} ( \overbrace{{G}_{x}^{2} {z}^{2} + {G}_{y}^{2} {z}^{2} + {G}_{z}^{2} \frac{{x}^{2} + {y}^{2}}{4}} \underbrace{- {G}_{x} {G}_{z} x z - {G}_{y} {G}_{z} y z} ) ,$$
wherein:
the terms Gₓ², G_{y}² and G_{z}² comprise self-squared terms,
the terms GₓG_{z} and G_{y}G_{z} comprise cross terms,
x, y and z represent coordinates in a 3D space,
B₀ represents a static magnetic field,
Gₓ, G_{y} and G_{z} represent applied magnetic gradients,
γ represents a gyromagnetic ratio characteristic of nuclei,
T represents a total time duration for applying the magnetic gradients; and
determine phase accruals φ_{xz}, φ_{yz} due to the self-squared terms of the concomitant field B_{c} and due to the cross terms of the concomitant field B_{c}, based on an encoding matrix that accounts for different possible sign combinations of the applied magnetic gradients Gₓ, G_{y} and G_{z}; and
generate a 2D or 3D image based upon one or more of the phase accruals φ_{xz}, φ_{yz}.
